(19) United States
(12) Patent Application Publication
(10) Pub. No.: US 2024/0266026 A1
(43) Pub. Date: Aug. 8, 2024

(54) INFORMATION PROCESSING APPARATUS, OPERATION METHOD OF INFORMATION PROCESSING APPARATUS, AND PROGRAM

(71) Applicant: FUJIFILM Corporation, Tokyo (JP)

(72) Inventor: Aya OGASAWARA, Tokyo (JP)

(73) Assignee: FUJIFILM Corporation, Tokyo (JP)

(21) Appl. No.: 18/421,939

(22) Filed: Jan. 24, 2024

(30) Foreign Application Priority Data
Feb. 2, 2023 (JP) 2023-014563

(51) Int. Cl. G16H 30/20; G16H 15/00

(52) U.S. Cl. CPC G16H 30/20; G16H 15/00

(57) ABSTRACT

Provided are an information processing apparatus, an operation method of the information processing apparatus, and a program that implement display control of a medical image according to a creation status of an interpretation report. An information processing apparatus receives a close instruction for one or more medical images displayed on a display device, collects status information indicating any of a first status, a second status, or a third status, the first status being a status according to a creation status of a latest interpretation report for a first medical image and indicating that the interpretation report has been created, the second status indicating that the interpretation report is incomplete, the third status corresponding to neither the first status nor the second status, and determines processing according to the status information for the first medical image.

### CROSS-REFERENCE TO RELATED APPLICATION

The present application claims priority under 35 U.S.C §119(a) to Japanese Patent Application No. 2023-014563 filed on Feb. 2, 2023, which is hereby expressly incorporated by reference, in its entirety, into the present application.

### BACKGROUND OF THE INVENTION

### 1. Field of the Invention

The present invention relates to an information processing apparatus, an operation method of an information processing apparatus, and a program.

### 2. Description of the Related Art

In a case where an image diagnostician interprets a target image or diagnoses a target image, the image diagnostician may display not only the target image but also a plurality of images such as a past image of a subject and images of other phases on a screen.

JP2016-202722A discloses a medical image display system that superimposes and displays past lesion information on a current image. JP2011-097983A discloses an image display system that acquires CAD information stored in association with a medical image and displays the acquired CAD information in a stepwise manner. In addition, CAD is an abbreviation for Computer-Aided Diagnosis.

### SUMMARY OF THE INVENTION

However, in a case where a large number of images are displayed, it may be difficult to determine which image is an image for which the creation of a report has been completed or which image is an image for which a report should be created from now. A user interface that distinguishes images using marks, colors, or the like for each image has a disadvantage that the marks are difficult to see due to small size and a disadvantage that a user needs to be aware of the meaning of the marks, the colors, and the like. The presence of the above-described disadvantages is a burden on a doctor or the like who is a user and is insufficient in terms of the functionality of the user interface.

In addition, in a case where the latest image is closed, there is a user interface in which a pop-up for confirming whether to close the latest image is displayed. However, such a user interface does not support a case where a plurality of images are compared and a report is created.

The system disclosed in JP2016-202722A assists in interpretation report (radiology report), but does not take into consideration a creation status of an interpretation report. The same applies to the system disclosed in JP2011-097983A.

The present invention has been made in view of such circumstances, and an object of the present invention is to provide an information processing apparatus, an operation method of an information processing apparatus, and a program that implement display control of a medical image according to a creation status of an interpretation report.

An information processing apparatus according to a first aspect of the present disclosure is an information processing apparatus comprising: one or more processors; and one or more memories in which instructions to be executed by the one or more processors are stored, in which the one or more processors are configured to: receive an instruction to close one or more medical images displayed on a display device; collect status information indicating any of a first status, a second status, or a third status, the first status being a status according to a creation status of a latest interpretation report for a first medical image that is a target of the instruction and indicating that the latest interpretation report has been created, the second status indicating that the latest interpretation report is incomplete, the third status corresponding to neither the first status nor the second status; and determine processing according to the status information for the first medical image.

With the information processing apparatus according to the first aspect of the present disclosure, status information indicating a status according to a creation status of an interpretation report is collected for an instruction target medical image that is a target of the close instruction, and processing is determined according to the collected status information. Accordingly, processing can be performed on the first medical image according to the creation status of the interpretation report.

The medical image displayed on the display device may include a past medical image that was captured and generated in the past than the medical image for which the latest interpretation report is to be created. Furthermore, the medical image displayed on the display device may include a medical image or the like referred to in the creation of the latest interpretation report.

The medical image displayed on the display device may be a two-dimensional reconstructed image or a three-dimensional reconstructed image. The two-dimensional reconstructed image may be a reconstructed image group including a plurality of two-dimensional reconstructed images.

The incompletion of the interpretation report may include a time before the latest interpretation report to be created is created.

With an information processing apparatus according to a second aspect, in the information processing apparatus according to the first aspect, the one or more processors may be configured to, in a case where the status information of the first medical image indicates the first status or the second status, execute processing of providing notification of a confirmation of whether to close the first medical image, as the processing according to the status information.

According to this aspect, it is possible to suppress the processing of erroneously closing the first medical images corresponding to the “interpretation report created” and the “interpretation report incomplete” during the creation of the latest interpretation report.

To provide notification of a confirmation of whether to close a target image, character information may be applied or sound or voice may be applied.

With an information processing apparatus according to a third aspect, in the information processing apparatus according to the first or second aspect, the one or more processors may be configured to, in a case where the status information of the first medical image indicates the first status or the second status, execute processing of continuing an open state of the first medical image, as the processing according to the status information.

According to this aspect, it is possible to suppress the processing of erroneously closing the first medical images corresponding to the “interpretation report created” and the “interpretation report incomplete” during the creation of the latest interpretation report.

With an information processing apparatus according to a fourth aspect, in the information processing apparatus according to any one of the first to third aspects, the one or more processors may be configured to, in a case where the status information of the first medical image indicates the third status, execute processing of closing the first medical image without performing processing of providing notification of a confirmation of whether to close the first medical image, as the processing according to the status information.

According to this aspect, reference images and the like that are not directly involved in creating the latest interpretation report are immediately closed in response to the close instruction.

With an information processing apparatus according to a fifth aspect, in the information processing apparatus according to any one of the first to fourth aspects, the one or more processors may be configured to receive an instruction to close an examination image group including the one or more medical images, as the instruction to close the one or more medical images displayed on the display device.

According to this aspect, the same processing as the instruction to close a medical image can be executed for an instruction to close an examination image group including one or more medical images.

With an information processing apparatus according to a sixth aspect, in the information processing apparatus according to the fifth aspect, the one or more processors may be configured to: in a case where the close instruction for the examination image group is received and the status information of the first medical image included in the examination image group indicates the first status or the second status, execute processing of providing notification of a confirmation of whether to close all the medical images included in the examination image group, as the processing according to the status information; and in a case where the close instruction for the examination image group is received and the status information of the first medical image included in the examination image group indicates the third status, execute processing of closing all the medical images included in the examination image group without performing the processing of providing notification of the confirmation of whether to close all the medical images, as the processing according to the status information.

According to this aspect, the same processing is performed on all medical images included in the examination image group that is the target of the closing processing.

With an information processing apparatus according to a seventh aspect, in the information processing apparatus according to the fifth or sixth aspect, the one or more processors may be configured to: in a case where the close instruction for the examination image group is received and the status information of the first medical image included in the examination image group indicates the first status or the second status, execute processing of continuing an open state of all the medical images included in the examination image group, as the processing according to the status information; and in a case where the close instruction for the examination image group is received and the status information of the first medical image included in the examination image group indicates the third status, execute processing of closing all the medical images included in the examination image group without performing processing of providing notification of a confirmation of whether to close all the medical images, as the processing according to the status information.

According to this aspect, the same processing is performed on all medical images included in the examination targeted for the closing processing.

With an information processing apparatus according to an eighth aspect, in the information processing apparatus according to any one of the first to seventh aspects, the one or more processors may be configured to determine the status of the first medical image based on information represented by a character string input in a case of creating the latest interpretation report.

According to this aspect, the status of the first medical image is determined according to the content of the latest interpretation report.

The character string may include one or more numbers.

With an information processing apparatus according to a ninth aspect, in the information processing apparatus according to the eighth aspect, the one or more processors may be configured to determine the status of the first medical image by applying a trained learning model that has been trained to output the status information of the first medical image in response to inputs of the interpretation report for the first medical image and the first medical image using the latest interpretation report, the first medical image, and the status information of the first medical image as learning.

According to this aspect, the processing time in the determination of the status is expected to be shortened, and the accuracy is expected to be improved.

With an information processing apparatus according to a tenth aspect, in the information processing apparatus according to any one of the first to ninth aspects, the one or more processors may be configured to: determine whether or not there is an interpretation report corresponding to the first medical image; in a case where there is an interpretation report corresponding to the first medical image, determine the status information for the first medical image to be the first status; and in a case where there is no interpretation report corresponding to the first medical image, determine the status information for the first medical image to be the second status.

According to this aspect, the status of the first medical image can be determined depending on the presence or absence of an interpretation report in the first medical image.

With an information processing apparatus according to an eleventh aspect, in the information processing apparatus according to any one of the first to tenth aspects, the one or more processors may be configured to: determine whether or not a character string is present in an interpretation report for an anatomical structure included in the first medical image; in a case where the character string is present in the interpretation report for the anatomical structure included in the first medical image, determine the status information for the first medical image to be the first status; and in a case where the character string is not present in the interpretation report for the anatomical structure included in the first medical image, determine the status information for the first medical image to be the second status.

According to this aspect, the status of the first medical image can be determined depending on the presence or absence of a sentence for each anatomical structure in the interpretation report.

With an information processing apparatus according to a twelfth aspect, in the information processing apparatus according to any one of the first to eleventh aspects, the one or more processors may be configured to: receive an input indicating completion of creation of an interpretation report; and in a case where an input indicating completion of an interpretation report corresponding to the first medical image is received, determine the status information for the first medical image to be the third status.

According to this aspect, the status of the first medical image can be determined depending on whether or not an input indicating completion of the interpretation report is received.

With an information processing apparatus according to a thirteenth aspect, in the information processing apparatus according to the twelfth aspect, the one or more processors may be configured to, in a case where it is determined that the status information for the first medical image indicates the third status, execute processing of closing the first medical image without performing processing of providing notification of a confirmation of whether to close the first medical image, as the processing according to the status information.

According to this aspect, it is possible to simplify the processing in response to the reception of the closing processing for the first medical image for which the interpretation report has been completed.

With an information processing apparatus according to a fourteenth aspect, in the information processing apparatus according to any one of the first to thirteenth aspects, the one or more processors may be configured to determine the status of the first medical image based on an examination date and time at which the first medical image was generated.

According to this aspect, in a case where the examination date and time at which the first medical image was generated is the latest, the status of the first medical image can be determined to be the first status or the second status. Further, in a case where the examination date and time at which the first medical image was generated is earlier than the latest examination date and time, the status information of the first medical image can be determined to be the third status.

An operation method of an information processing apparatus according to a fifteenth aspect of the present disclosure is an operation method of an information processing apparatus that performs information processing for a medical image to which a computer is applied, the operation method comprising: by the information processing apparatus, receiving a close instruction for one or more medical images displayed on a display device; collecting status information indicating any of a first status, a second status, or a third status, the first status being a status according to a creation status of a latest interpretation report for a first medical image that is a target of the instruction and indicating that the interpretation report has been created, the second status indicating that the interpretation report is incomplete, the third status corresponding to neither the first status nor the second status; and determining processing according to the status information for the first medical image.

With the operation method of the information processing apparatus according to the fifteenth aspect of the present disclosure, it is possible to obtain the same effects as the information processing apparatus according to the first aspect of the present disclosure.

In the operation method of the information processing apparatus according to the fifteenth aspect, the same items as those specified in the second to fourteenth aspects can be combined as appropriate. In that case, the component responsible for the specified processing or function in the information processing apparatus can be understood as a component of the operation method of the information processing apparatus responsible for the corresponding processing or function.

A program according to a sixteenth aspect of the present disclosure is a program causing a computer to implement: a function of receiving a close instruction for one or more medical images displayed on a display device; a function of collecting status information indicating any of a first status, a second status, or a third status, the first status being a status according to a creation status of an interpretation report for a first medical image that is a target of the instruction and indicating that the interpretation report has been created, the second status indicating that the interpretation report is incomplete, the third status corresponding to neither the first status nor the second status; and a function of determining processing according to the status information for the first medical image.

With the program according to the sixteenth aspect of the present disclosure, it is possible to obtain the same effects as the information processing apparatus according to the first aspect of the present disclosure.

In the program according to the sixteenth aspect, the same items as those specified in the second to fourteenth aspects can be combined as appropriate. In that case, the component responsible for the specified processing or function in the information processing apparatus can be understood as a component of the program responsible for the corresponding processing or function.

According to the aspects of the present invention, status information indicating a status according to a creation status of an interpretation report is collected for the first medical image that is the target of the close instruction, and processing is determined according to the collected status information. Accordingly, processing can be performed on the first medical image according to the creation status of the interpretation report.

### BRIEF DESCRIPTION OF THE DRAWINGS

FIG. 1 is an overall configuration diagram of a hospital information system.

FIG. 2 is a functional block diagram showing an electric configuration of a server device shown in FIG. 1.

FIG. 3 is a block diagram schematically showing an example of a hardware configuration of the server device shown in FIG. 2.

FIG. 4 is a flowchart showing a procedure of an information processing method according to an embodiment.

FIG. 5 is a diagram showing an example of a display screen applied to display of a medical image.

FIG. 6 is a schematic diagram showing a configuration example of an examination image group.

FIG. 7 is a schematic diagram showing a first display example of a plurality of medical images.

FIG. 8 is a schematic diagram showing a second display example of a plurality of medical images.

FIG. 9 is a schematic diagram showing a third display example of a plurality of medical images.

FIG. 10 is a diagram showing another example of a display screen on which a plurality of medical images are displayed.

FIG. 11 is a diagram showing an example of an interpretation report.

FIG. 12 is a schematic diagram showing an example of a pop-up.

FIG. 13 is a schematic diagram showing a first example of a creation status of an interpretation report.

FIG. 14 is a schematic diagram showing a second example of a creation status of an interpretation report.

FIG. 15 is a schematic diagram showing a third example of a creation status of an interpretation report.

FIG. 16 is a schematic diagram showing a fourth example of a creation status of an interpretation report.

FIG. 17 is a flowchart showing a procedure of a status information collection method.

FIG. 18 is a schematic diagram showing a modification example of status information collection.

FIG. 19 is a schematic diagram of a learning model that performs status information collection.

FIG. 20 is a schematic diagram showing a first example of determining processing for a close instruction.

FIG. 21 is a schematic diagram showing an example of a pop-up.

FIG. 22 is a schematic diagram showing another example of a pop-up.

FIG. 23 is a schematic diagram showing a second example of determining processing for a close instruction.

FIG. 24 is a schematic diagram showing a third example of determining processing for a close instruction.

### DESCRIPTION OF THE PREFERRED EMBODIMENTS

Hereinafter, a preferred embodiment of the present invention will be described in detail with reference to the accompanying drawings. In the present specification, the same components are denoted by the same reference numerals, and duplicate description thereof will be omitted as appropriate.

### Configuration Example of Hospital Information System

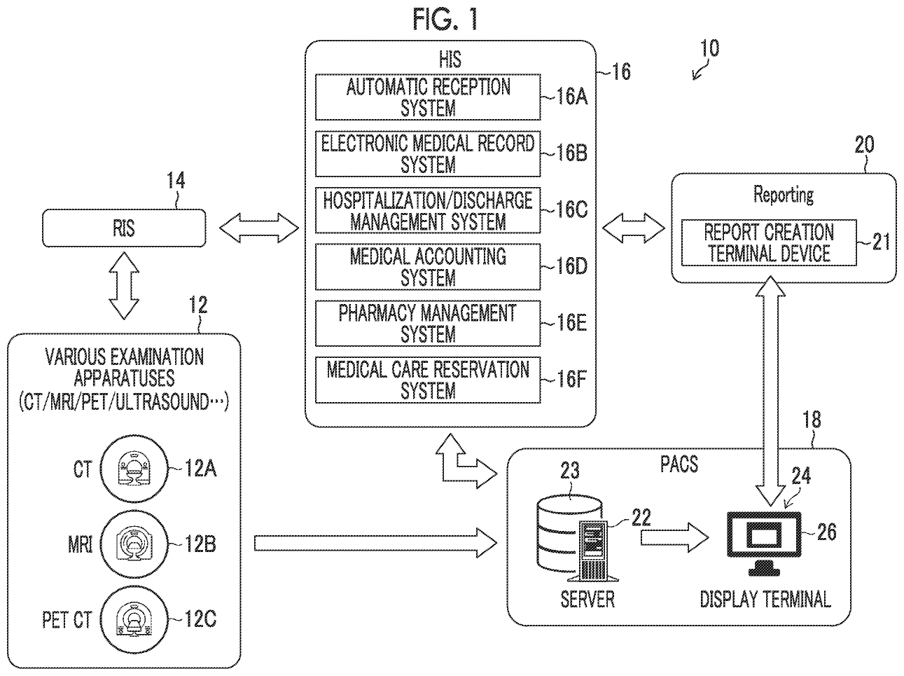

FIG. 1 is an overall configuration diagram of a hospital information system. Various systems constituting a hospital system 10 shown in FIG. 1 include one or more computers, and the one or more computers execute various programs stored on tangible computer-readable mediums to implement one or more functions in the various systems.

The hospital system 10 comprises various examination apparatuses 12. The examination apparatus 12 images an examination target subject, which is a subject, and generates a medical image. In FIG. 1, a CT apparatus 12A, an MRI apparatus 12B, and a PETCT apparatus 12C are exemplified as the examination apparatus 12. Other examples of the examination apparatus 12 include an X-ray imaging apparatus, an ultrasound apparatus, a CR apparatus using a flat X-ray detector, and the like.

Note that, CT is an abbreviation for Computed Tomography. MRI is an abbreviation for Magnetic Resonance Imaging. PET is an abbreviation for Positron Emission Tomography. CR is an abbreviation for Computed Radiography. The examination apparatus 12 may also be referred to as a modality.

The hospital system 10 comprises a radiology information system 14. The radiology information system 14 mainly performs management from reservation of examinations and treatments in which radiation equipment is used to examination results. The radiology information system 14 stores medical images generated in examinations, past interpretation reports, current interpretation reports created in a current examination, and the like. The radiology information system 14 may be represented using RIS, which is an abbreviation for Radiology Information Systems.

The hospital system 10 comprises a hospital information system 16. The hospital information system 16 is a general term for various information systems in a hospital. In general, the hospital information system 16 may include an automatic reception system 16A, an electronic medical record system 16B, a hospitalization/discharge management system 16C, a medical accounting system 16D, a pharmacy management system 16E, a medical care reservation system 16F, and the like.

The hospital information system 16 stores all information regarding the hospital. For example, the electronic medical record system 16B stores a surgical history, a treatment history, and the like of each patient as an electronic medical record. The hospital information system 16 may be represented using an abbreviation HIS for Hospital Information Systems.

The hospital system 10 comprises an image storage communication system 18. The image storage communication system 18 comprises a server device 22 and a display terminal 24. The server device 22 is provided with a database 23. The display terminal 24 is provided with a display device 26.

The image storage communication system 18 receives a medical image transmitted from the examination apparatus 12. The received medical image is stored in the database 23. The image storage communication system 18 reads out the medical image stored in the database 23. The read-out medical image is displayed on the display device 26 of the display terminal 24. DICOM is applied as a standard for storage and communication of medical images managed using the server device 22, the database 23, and the display terminal 24. DICOM is an abbreviation for Digital Imaging and COmmunications in Medicine. The server device 22 may be referred to as a DICOM server.

A radiologist can visually recognize the medical image to be diagnosed using the display terminal 24. In addition, the radiologist can check the content of the orders transmitted from various medical departments using the display terminal 24. Furthermore, the radiologist can ascertain the purpose of the examination based on the orders transmitted from various medical departments. In addition, the image storage communication system 18 may be represented using PACS, which is an abbreviation for Picture Archiving and Communication Systems.

The hospital system 10 comprises a report creation system 20. The report creation system 20 comprises a report creation terminal device 21 that is operated by a radiologist to create an interpretation report. The report creation terminal device 21 may store a program for supporting creation of the interpretation report, and execute the program for supporting the creation of the interpretation report.

The examination apparatus 12 and the like constituting the hospital system 10 are electrically connected to each other via an in-hospital network in a communicable manner. An in-hospital network LAN can be applied. The in-hospital network may be wired or wireless.

The in-hospital network may be connected to a public line network such as the Internet via a router (not shown). That is, the hospital system 10 may be electrically connected to an external device, such as a cloud server, via an in-hospital network and a public line network in a communicable manner. LAN is an abbreviation for Local Area Network.

### Configuration Example of Server Device Provided in Image Storage Communication System

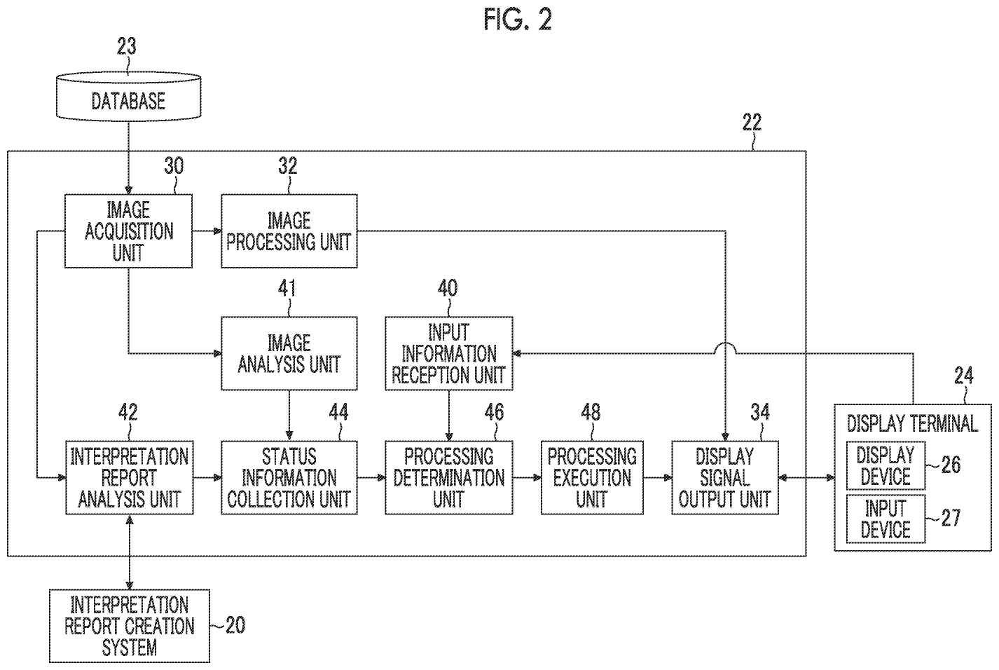

FIG. 2 is a functional block diagram showing an electric configuration of the server device shown in FIG. 1. A computer is applied to the server device 22. The server device 22 executes a prescribed program to implement various functions. The server device 22 may be a physical server or may be a virtual server.

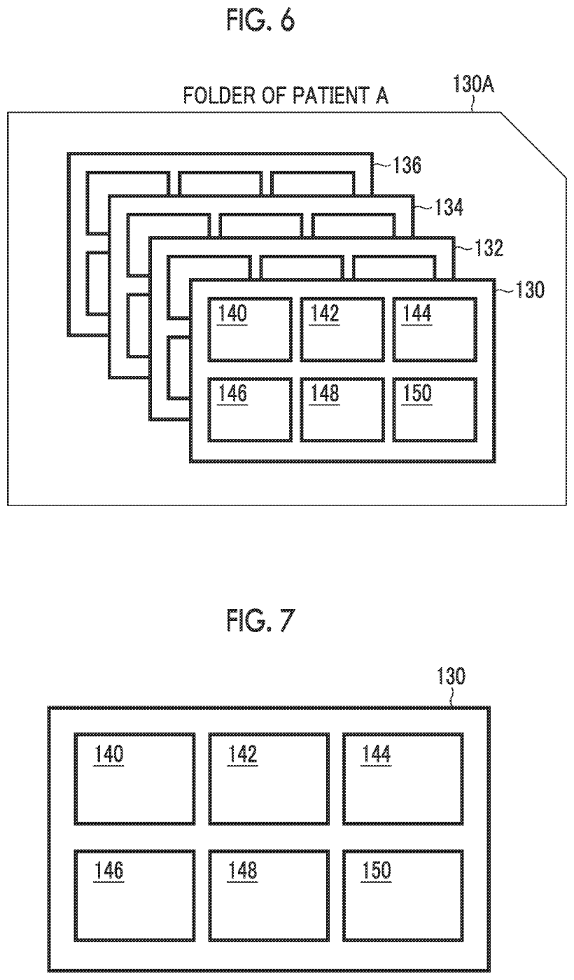

The server device 22 comprises an image acquisition unit 30. The image acquisition unit 30 acquires a medical image stored in the database 23. The image acquisition unit 30 may 30 may acquire an examination image group including one or more medical images. The examination image group is shown in FIG. 6 using reference numeral 130 and the like.

The server device 22 comprises an image processing unit 32. The image processing unit 32 performs various types of image processing on the medical image acquired using the image acquisition unit 30. Examples of various types of image processing include processing corresponding to a display condition of the display device 26, such as resolution conversion processing and size conversion processing. The server device 22 comprises a display signal output unit 34. The display signal output unit 34 transmits a display signal representing the medical image subjected to image processing using the image processing unit 32 to the display device 26 of the display terminal 24.

The server device 22 comprises an input information reception unit 40. The input information reception unit 40 receives an input signal representing input information according to the operation of the input device 27 of the display terminal 24. A keyboard, a mouse, a touch panel type display device 26, or the like may be applied to the input device 27. Examples of input information include input information indicating that an instruction to close the medical image displayed on the display device 26 of the display terminal 24 has been given.

The server device 22 comprises an image analysis unit 41. The image analysis unit 41 analyzes the medical image acquired using the image acquisition unit 30. Examples of analysis of the medical image include analysis of an examination date and time, analysis of an examination apparatus, analysis of an organ included in a medical image to be analyzed, and the like.

Analysis of the examination date and time and analysis of the examination apparatus can be performed based on accessory information attached to the medical image to be analyzed. Analysis of an organ included in a medical image to be analyzed can be performed by applying region-of-interest extraction processing. Note that in a case where a plurality of organs are included in the medical image to be analyzed, each of the plurality of organs is extracted.

A trained learning model may be applied to the image analysis unit 41. The learning model applied to the image analysis unit 41 is a recognition model that recognizes one or more anatomical structures from one medical image, and a learning model that outputs an anatomical structure recognition result in a case where a medical image is input, using a set of the medical image and the anatomical structure recognition result as learning data, may be applied. Deep learning may be applied to the training of the learning model applied to the image analysis unit 41.

The server device 22 comprises an interpretation report analysis unit 42. The interpretation report analysis unit 42 analyzes the creation status of the interpretation report for the medical image to be interpreted with reference to the report creation system 20 for the medical image acquired using the image acquisition unit 30. Examples of analysis of an interpretation report include keyword extraction, which extracts keywords such as preset terms such as a date and time, an examination apparatus name, an organ name, and a comparison.

For example, in a case of analyzing an interpretation report of the abdomen, the interpretation report analysis unit 42 analyzes whether or not all organs included in the abdomen, such as the liver, gallbladder, pancreas, and spleen, are described.

The interpretation report analysis unit 42 may analyze a correspondence relationship between a result of CAD of the medical image for which the interpretation report is to be created and content described in the interpretation report. For example, in a case where a lung nodule is described in the result of CAD, it may be analyzed whether or not there is a description regarding the lung in the interpretation report.

A trained learning model may be applied to the interpretation report analysis unit 42. As the learning model applied to the interpretation report analysis unit 42, an extraction model that extracts specific terms and the like such as an examination date and time, an anatomical structure of an organ or the like, and a comparison in a case where one or more sentences of the interpretation report are input may be applied. As the learning model applied to the interpretation report analysis unit 42, a learning model that outputs a predetermined character string such as an examination date and time in a case where a sentence included in the interpretation report is input, using a set of the sentence included in the interpretation report and the examination date and time to be extracted and the like as learning data, may be applied. Deep learning may be applied to the training of the learning model applied to the image analysis unit 41.

The server device 22 comprises a status information collection unit 44. The status information collection unit 44 collects status information indicating a status according to the creation status of the interpretation report for the medical image acquired using the image acquisition unit 30 based on the analysis result of the medical image and the analysis result of the interpretation report. Note that the details of the status for each medical image will be described later.

The server device 22 comprises a processing determination unit 46 and a processing execution unit 48. The processing determination unit 46 determines processing to be performed on an instruction target medical image for which a close instruction has been given, based on the status information for each medical image. The processing execution unit 48 executes the processing on the instruction target medical image determined by the processing determination unit 46. The processing execution unit 48 reflects the processing on the medical image displayed on the display device 26 via the display signal output unit 34. The instruction target medical image described in the embodiment is an example of a first medical image.

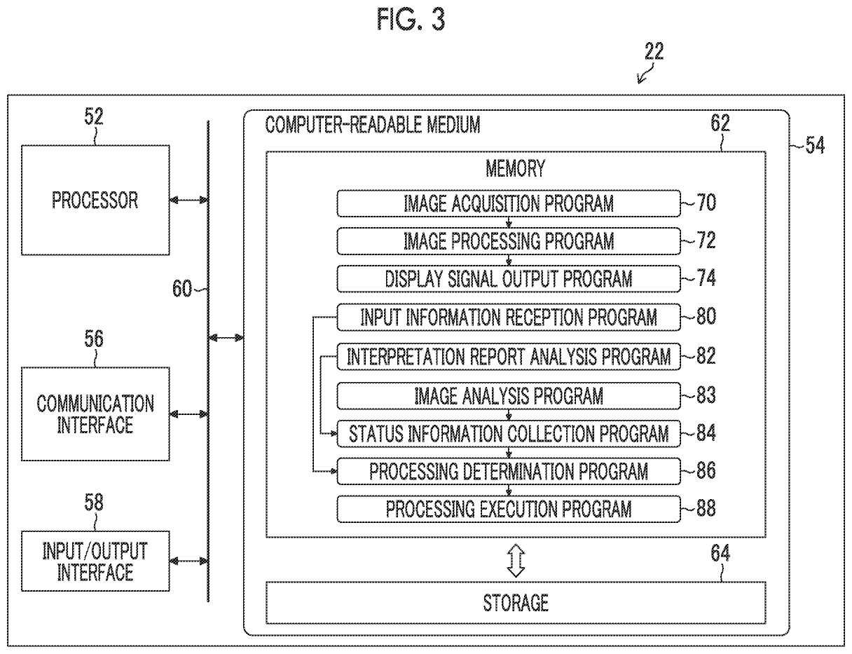

FIG. 3 is a block diagram schematically showing an example of a hardware configuration of the server device shown in FIG. 2. The server device 22 comprises one or more processors 52 and one or more memories 62. The server device 22 comprises a communication interface 56 and an input/output interface 58.

The processor 52 executes various programs stored in the memory 62 of a computer-readable medium 54 to implement various functions of the server device 22. The processor 52 includes a central processing unit (CPU). The processor 52 may include a graphics processing unit (GPU). The processor 52 is connected to the computer-readable medium 54, the communication interface 56, and the input/output interface 58 via a bus 60.

The computer-readable medium 54 includes a memory 62 which is a main memory and a storage 64 which is an auxiliary memory. A semiconductor memory, a hard disk apparatus, a solid-state drive apparatus, and the like may be applied to the computer-readable medium 54. Any combination of a plurality of devices may be applied to the computer-readable medium 54.

A hard disk apparatus may be referred to as an HDD, which is an abbreviation for Hard Disk Drive in English. A solid-state drive apparatus may be referred to as an SSD, which is an abbreviation for Solid-State Drive in English.

The memory 62 of the computer-readable medium 54 stores an image acquisition program 70, an image processing program 72, and a display signal output program 74. In addition, the memory 62 stores an input information reception program 80, an interpretation report analysis program 82, an image analysis program 83, a status information collection program 84, a processing determination program 86, and a processing execution program 88.

The image acquisition program 70 is applied to the image acquisition unit 30 shown in FIG. 2 to implement an image acquisition function. The image processing program 72 is applied to the image processing unit 32 to implement an image processing function using a processing result of the image acquisition program 70. The display signal output program 74 is applied to the display signal output unit 34 to implement a display signal output function using a processing result of the image processing program 72.

The input information reception program 80 is applied to the input information reception unit 40 to implement an input information reception function. The interpretation report analysis program 82 is applied to the interpretation report analysis unit 42 to implement an interpretation report analysis function. The image analysis program 83 is applied to the image analysis unit 41 to implement an image analysis function.

The status information collection program 84 is applied to the status information collection unit 44 to implement a status information collecting function using a processing result of the interpretation report analysis program 82 and a processing result of the image analysis program 83. The processing determination program 86 is applied to the processing determination unit 46 to implement a processing determination function using a processing result of the input information reception program 80 and a processing result of the status information collection program 84. The processing execution program 88 is applied to the processing execution unit 48 to implement a processing execution function using a processing result of the processing determination program 86.

Various programs stored in the computer-readable medium 54 include one or more instructions. The computer-readable medium 54 stores various types of data, various parameters, and the like. Note that the term “program” is synonymous with the term “software”.

The hardware structure of the processor 52 is various processors as shown below. The various processors include a central processing unit (CPU) that is a general-purpose processor that acts as various functional units by executing software (programs), a programmable logic device (PLD) that is a processor whose circuit configuration can be changed after manufacture, such as a graphics processing unit (GPU) and a field-programmable gate array (FPGA) which are processors specialized for image processing, a dedicated electrical circuit that is a processor having a circuit configuration designed exclusively for executing specific processing such as an application-specific integrated circuit (ASIC), and the like.

One processing unit may be configured by one of the various processors, or may be configured by the same or different types of two or more processors (for example, a plurality of FPGAs, a combination of a CPU and an FPGA, or a combination of a CPU and a GPU). In addition, a plurality of functional units may be configured by one processor. As an example of configuring a plurality of functional units via one processor, first, as represented by a computer, such as a client or a server, there is a form in which one processor is configured by a combination of one or more CPUs and software, and this processor acts as a plurality of functional units. Second, as represented by a system-on-chip (SoC) or the like, there is a form of using a processor for implementing the function of the entire system including a plurality of functional units with one integrated circuit (IC) chip. Thus, various functional units are configured by using one or more of the above-described various processors as hardware structures.

Furthermore, the hardware structure of these various processors is, more specifically, an electrical circuit (circuitry) in which circuit elements such as semiconductor elements are combined.

The memory 62 stores instructions for execution by the processor 52. The memory 62 includes a random-access memory (RAM) and a read-only memory (ROM)(which are not shown). The processor 52 uses the RAM as a work area, executes software using parameters and various programs including an image processing program including the image acquisition program 70 and the like stored in the ROM, and uses the parameters stored in the ROM or the like to execute various types of processing of the server device 22.

The display terminal 24 shown in FIG. 1 may implement an information processing function in the server device 22. That is, the display terminal 24 may comprise various processing units such as the image acquisition unit 30 shown in FIG. 3 to implement functions of various processing units. In addition, the hospital system 10 shown in FIG. 1 may be provided with an information processing apparatus comprising various processing units such as the image acquisition unit 30 shown in FIG. 3, in addition to the server device 22 and the display terminal 24.

### Procedure of Information Processing Method According to Embodiment

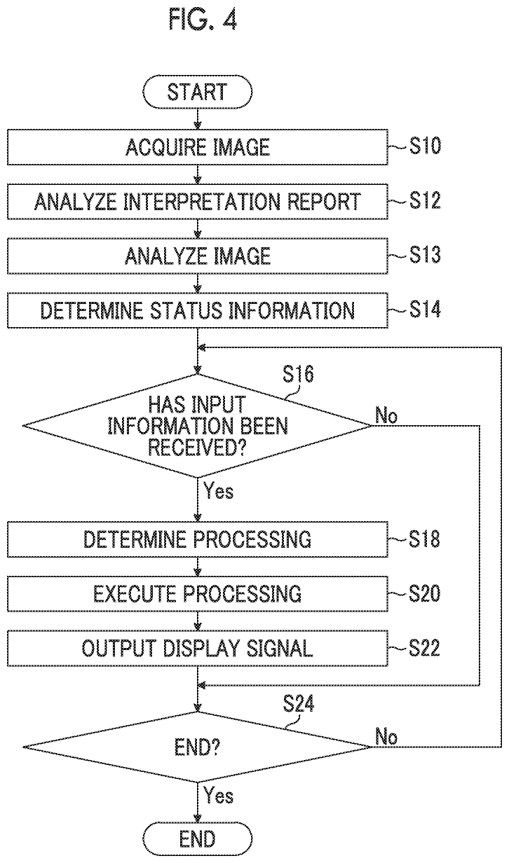

FIG. 4 is a flowchart showing a procedure of an information processing method according to an embodiment. The information processing method described below is understood as an operation method of an information processing apparatus to which a computer including one or more processors is applied.

In an image acquisition step S10, the image acquisition unit 30 shown in FIG. 2 acquires one or more medical images from the database 23. In the image acquisition step S10, a desired medical image can be acquired from the database 23 in response to an operation by an operator of the display terminal 24.

The medical image acquired in the image acquisition step S10 is subjected to image processing such as processing corresponding to the display condition of the display device 26 shown in FIG. 1, is converted into a display signal, and is then transmitted to the display device 26. The display device 26 displays a medical image corresponding to the received display signal. After the image acquisition step S10, the process proceeds to an interpretation report analysis step S12.

In the interpretation report analysis step S12, the interpretation report analysis unit 42 analyzes the creation status of the interpretation report in the medical image acquired in the image acquisition step S10 with reference to the report creation system 20. In the interpretation report analysis step S12, the analysis result of the interpretation report may be stored. After the interpretation report analysis step S12, the process proceeds to an image analysis step S13.

In the image analysis step S13, the image analysis unit 41 analyzes the medical image acquired in the image acquisition step S10. In the image analysis step S13, the analysis result of the medical image may be stored. After the image analysis step S13, the process proceeds to a status information collection step S14. Note that the interpretation report analysis step S12 and the image analysis step S13 may be executed in parallel, or the order may be switched.

In the status information collection step S14, the status information collection unit 44 collects status information indicating the status of each medical image acquired in the image acquisition step S10 based on the creation status of the interpretation report for each medical image and the analysis result of the medical image. After the status information collection step S14, the process proceeds to an input information reception determination step S16.

In the input information reception determination step S16, the input information reception unit 40 determines whether or not input information corresponding to processing for closing the medical image displayed on the display device 26 has been received. In a case where the input information reception unit 40 determines that the input information has not been received, a No determination is made. In a case where the No determination is made, the process proceeds to an end determination step S24. In a case where the No determination is made, waiting for the reception of the input information is carried out, and in a case where the input information has not been received even after a certain period of time has elapsed, the process may proceed to the end determination step S24.

On the other hand, in the input information reception determination step S16, in a case where the input information reception unit 40 determines that the input information has been received, a Yes determination is made. In a case where the Yes determination is made, the process proceeds to a processing determination step S18.

In the input information reception determination step S16, the image acquisition step S10, the interpretation report analysis step S12, and the status information collection step S14 may be newly executed during a period of waiting for the reception of the input information.

In the processing determination step S18, the processing determination unit 46 determines processing to be performed on the medical image for which input information of an instruction for the closing processing has been received, based on the status of the medical image collected in the status information collection step S14. After the processing determination step S18, the process proceeds to a processing execution step S20.

In the processing execution step S20, the processing execution unit 48 executes the processing on the medical image for which the input information of the instruction for the closing processing has been received. After the processing execution step S20, the process proceeds to a display signal output step S22.

In the display signal output step S22, the display signal output unit 34 outputs a display signal corresponding to the processing performed on the medical image displayed on the display device 26. The display device 26 executes display corresponding to the processing performed on the medical image. Example of the display corresponding to the processing performed on the medical image includes a pop-up display for confirming whether to close the medical image. After the display signal output step S22, the process proceeds to the end determination step S24.

In the end determination step S24, the server device 22 performs the end determination of the information processing method. That is, in the end determination step S24, the server device 22 determines whether or not an end condition of the information processing method is satisfied.

In the end determination step S24, in a case where the server device 22 determines that the end condition of the information processing method is not satisfied, a No determination is made. In a case where the No determination is made, the process proceeds to the input information reception determination step S16, and the steps from the input information reception determination step S16 to the end determination step S24 are repeatedly executed until a Yes determination is made in the end determination step S24.

On the other hand, in the end determination step S24, in a case where the server device 22 determines that the end condition of the information processing method is satisfied, a Yes determination is made. In a case where the Yes determination is made, prescribed end processing is executed, and the server device 22 ends the information processing method. Examples of an end condition of the information processing method include the end of creating an interpretation report for the medical image displayed on the display device 26 of the display terminal 24.

### Specific Example of Display Screen of Medical Image

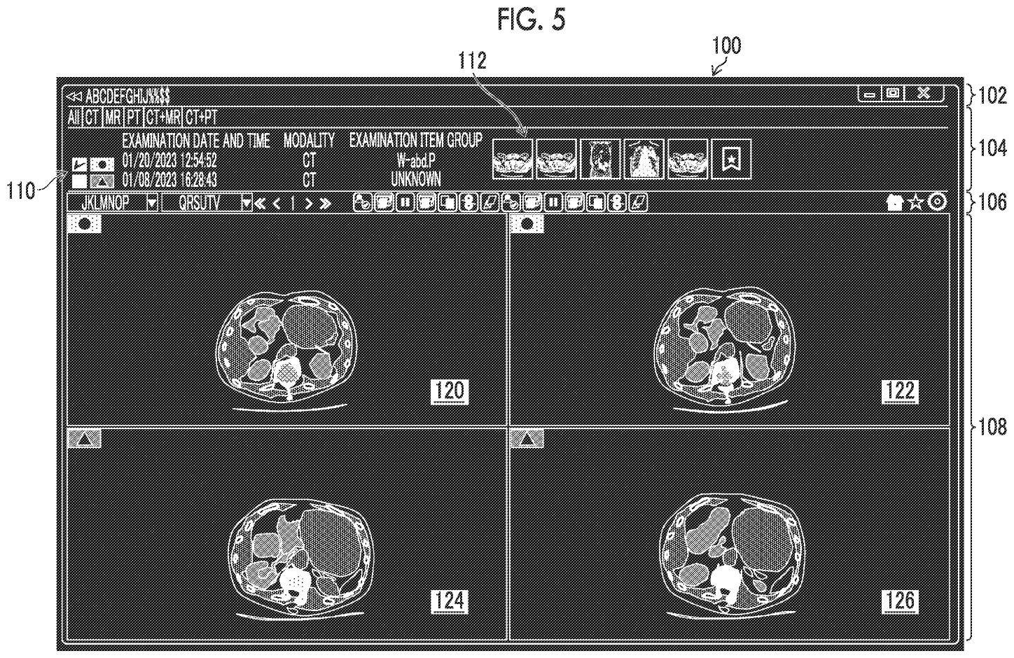

FIG. 5 is a diagram showing an example of a display screen applied to display of a medical image. FIG. 5 shows a display screen 100 on which four medical images are displayed. Note that the display screen 100 may be configured as a window displayed on the display device 26 shown in FIG. 1. In FIG. 5, any character is shown as an example of the character. In addition, as the character string, a character string including a plurality of any characters is shown.

The display screen 100 includes a title bar 102 on which a title of a screen is displayed, an information bar 104 on which various types of information are displayed, a toolbar 106 on which icons corresponding to various tools are displayed, and an image display region 108 on which medical images are displayed.

The information bar 104 displays an examination image group list 110 including the medical images displayed in the image display region 108. The examination image group displayed in the examination image group list 110 corresponds to an examination image group including the medical images displayed in the image display region 108.

Further, the information bar 104 displays thumbnail images 112 of the medical images included in any one examination image group. The thumbnails displayed on the information bar 104 correspond to medical images included in the medical image group selected in the examination image group list 110.

The image display region 108 displays one or more medical images. FIG. 5 shows an example in which two medical images 120 and 122 included in a first examination image group are displayed and two medical images 126 and 128 included in a second examination image group are displayed. Note that although FIG. 5 shows an example in which the image display region 108 is divided into four regions, the number of divisions of the image display region 108 can be defined according to the number of medical images to be displayed.

A first examination and a second examination are examinations performed on the same examination target subject at different dates and times. For example, the first examination is the latest examination, and examples of the first examination include the current examination. The first examination image group includes one or more medical images generated by performing the first examination.

The second examination is a past examination performed before the first examination, and examples of the second examination include the previous examination. The second examination image group includes one or more medical images generated by performing the second examination.

Although FIG. 5 shows, as an example, a case where two examination image groups are displayed, the number of displayed examination image groups need only be one or more, and the number of displayed examination image groups may be three or more. In a case where a plurality of examination image groups are displayed, a combination of the latest examination and one or more past examinations may be used.

### Specific Example of Management of Medical Image

FIG. 6 is a schematic diagram showing a configuration example of an examination image group. FIG. 6 schematically shows a configuration example of a folder of any patient A. As shown in FIG. 6, a folder 130A is generated for each patient. The folder 130A includes one or more examination image groups. FIG. 6 shows an example in which four examination image groups including an examination image group 130, an examination image group 132, an examination image group 134, and an examination image group 136 are stored in the folder 130A.

FIG. 6 shows an example in which an examination date and time of each of the examination image group 130, the examination image group 132, the examination image group 134, and the examination image group 136 is different. The examination image group 130 and the like include one or more medical images. For example, the examination image group 130 includes a medical image 140, a medical image 142, a medical image 144, a medical image 146, a medical image 148, and a medical image 150.

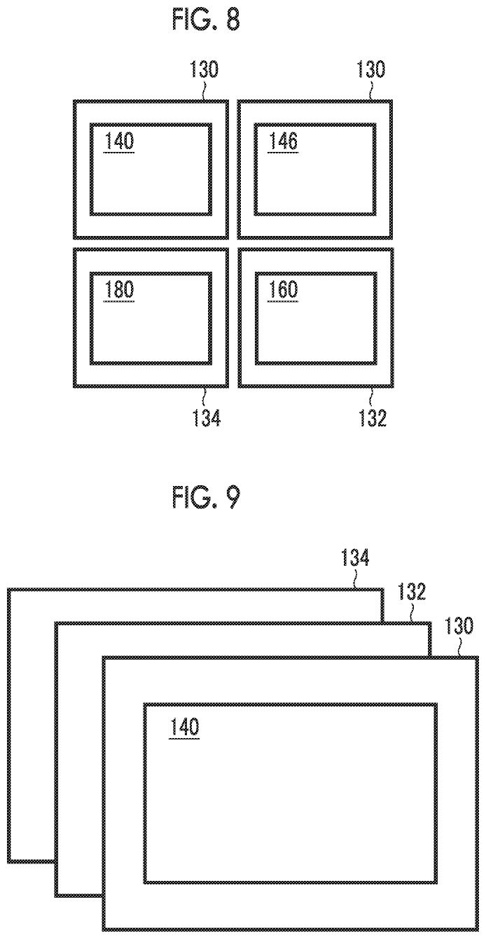

FIG. 7 is a schematic diagram showing a first display example of a plurality of medical images. FIG. 7 schematically shows a layout of a plurality of medical images displayed in the image display region 108 shown in FIG. 5. The same applies to FIGS. 8 and 9.

In the example of the layout of a plurality of medical images shown in FIG. 7, the examination image group 130 is displayed, and a medical image 140, a medical image 142, a medical image 144, a medical image 146, a medical image 148, and a medical image 150 included in the examination image group 130 are displayed. That is, one or more medical images displayed in the image display region 108 shown in FIG. 5 may include a plurality of medical images in the same examination image group.

FIG. 8 is a schematic diagram showing a second display example of a plurality of medical images. In the example of the layout of a plurality of medical images shown in FIG. 8, a medical image 140 included in the examination image group 130, a medical image 146 included in the examination image group 130, a medical image 160 included in the examination image group 132, and a medical image 180 included in the examination image group 134 are displayed. That is, the one or more medical images displayed in the image display region 108 shown in FIG. 5 may be medical images included in a plurality of different examination image groups, or may be a plurality of medical images in one examination image group. In the example shown in FIG. 8, a display mode with a different background color or the like may be applied to each of different medical image groups.

FIG. 9 is a schematic diagram showing a third display example of a plurality of medical images. In the example of the layout of a plurality of medical images shown in FIG. 9, a case is shown in which a medical images 140 included in the examination image group 130 is displayed and the examination image group 132 and the examination image group 134 are stored. For example, FIG. 9 corresponds to a case where there are a plurality of examination image groups, the medical image that should originally be displayed is not displayed, and the medical images included in the past examination image group are displayed.

In a case where one or more medical images shown in FIGS. 7 to 9 are displayed, it may be difficult to ascertain which image to close or which examination image group to close. Therefore, in a case where an instruction to close a medical image is received or in a case where an instruction to close an examination image group is received, processing for a close instruction according to the creation status of the interpretation report for each medical image is determined.

### Specific Example of Information Processing Method

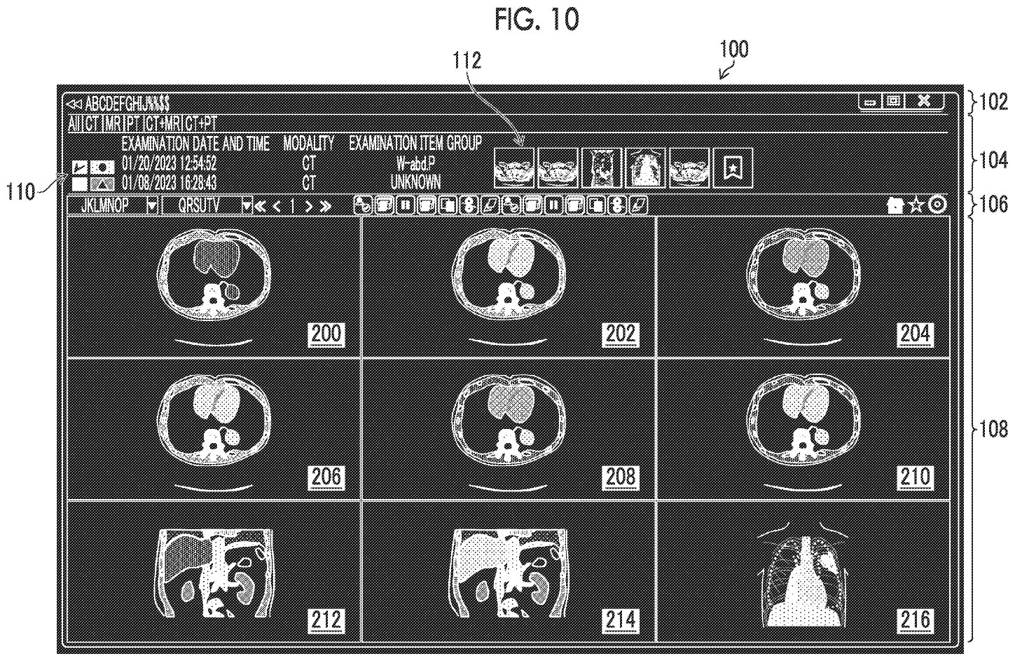

FIG. 10 is a diagram showing another example of a display screen on which a plurality of medical images are displayed. FIG. 10 shows an example in which nine medical images are displayed. In FIG. 10, two-dimensional reconstructed images representing axial cross sections are shown as a medical image 200, a medical image 202, a medical image 204, a medical image 206, a medical image 208, and a medical image 210. In addition, in FIG. 10, two-dimensional reconstructed images representing a coronal cross section are shown as a medical image 212 and a medical image 214. Further, in FIG. 10, a two-dimensional reconstructed image representing a sagittal cross section is shown as a medical image 216.

The medical image 200 shown in FIG. 10 is the latest image generated in the latest examination. The latest image may be a current image generated in the current examination. Each of the medical image 202 to the medical image 216 is a past image generated in the past examination.

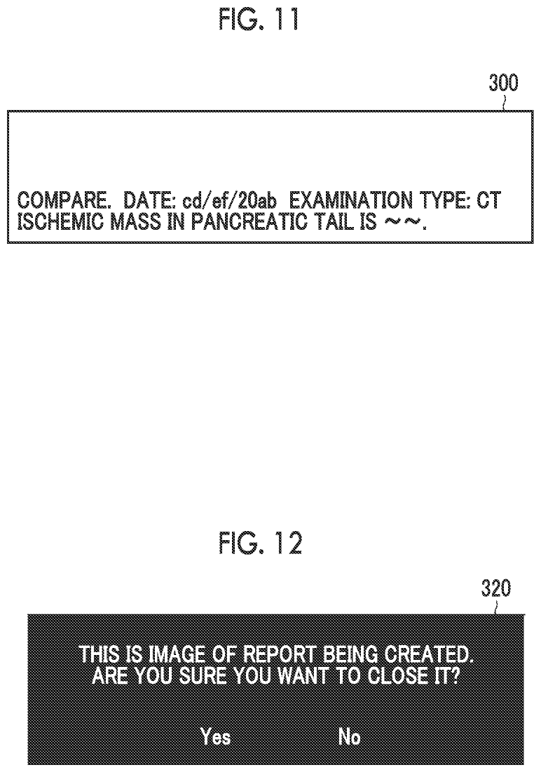

FIG. 11 is a diagram showing an example of an interpretation report. FIG. 11 shows a part of an interpretation report 300 created in a case where the medical image 200 shown in FIG. 10 is an interpretation target. The interpretation report 300 includes a description indicating that the medical image 200, which is the latest image, is compared with the past image. Furthermore, in the interpretation report 300, cd/ef/20ab is described as the date of the past examination for which the past image was generated. Note that a to f represent any numbers.

In this case, the medical image 200 to be interpreted corresponds to a first status indicating that the latest interpretation report has been created or corresponds to a second status indicating that the latest interpretation report is incomplete.

A past image corresponding to the date of the past examination described above is assumed to be a medical image 208. Although the medical image 208, which is a past image, is not an interpretation target in the latest examination, it is a medical image as a comparison target in the interpretation report 300 shown in FIG. 11. Then, similarly to the medical image 200, the medical image 208 corresponds to the first status or the second status.

On the other hand, a past medical image, such as the medical image 202 that does not correspond to the comparison target in the interpretation report 300 in the latest examination, corresponds to a third status indicating that the medical image corresponds to neither the first status nor the second status.

Examples of a medical image corresponding to the third status include a past image that is displayed simply as a reference, a past image that is determined to be normal in a past examination, a past image in which none of detection results of the CAD is obtained in a past examination, and the like.

Another example of a medical image corresponding to the third status includes a past image generated by imaging a part different from the part that should be reported in the latest interpretation report. For example, in the case of creating a report of the abdomen in the latest interpretation report, it is considered that no problem will occur even in a case where the latest image of the head and the past image of the head are closed.

FIG. 12 is a schematic diagram showing an example of a pop-up. In a case where a close instruction for the medical image 200 shown in FIG. 10 is received, processing of displaying a pop-up 320 for confirming whether to close the medical image 200 is executed before processing of closing the medical image 200 is executed. The same applies to the medical image 208.

In a case where the pop-up 320 shown in FIG. 12 is displayed on the display device 26 shown in FIG. 1, the operator, such as a doctor, can select Yes in a case where it is good to close the image, and select No in a case where the image is not to be closed. That is, in a case where an important image related to the creation of the latest interpretation report is likely to be closed, the pop-up 320 for confirming whether to close the image is displayed, and thus the occurrence of omissions and errors in the interpretation report is suppressed.

On the other hand, in a case where a close instruction for the medical image 202 or the like corresponding to the third status is received, processing of closing the medical image 202 or the like without displaying the pop-up 320 for confirming whether to close the medical image 202 or the like is executed. The medical image 202 or the like of which third status information has been acquired is defined as an unimportant image having a low involvement in the creation of the latest interpretation report, and processing of quickly closing the medical image 202 or the like in response to a close instruction is executed.

Although FIG. 12 shows, as an example, the pop-up 320 in which a notification is executed using character information as an aspect of notification in a case where a confirmation of whether to close a target image is made, as the notification in a case where the confirmation of whether to close a target image is made, a sound such as a beep sound may be used, or a voice for confirming whether to close a target image may be used.

### Correspondence Relationship Between Creation Status of Interpretation Report and Status

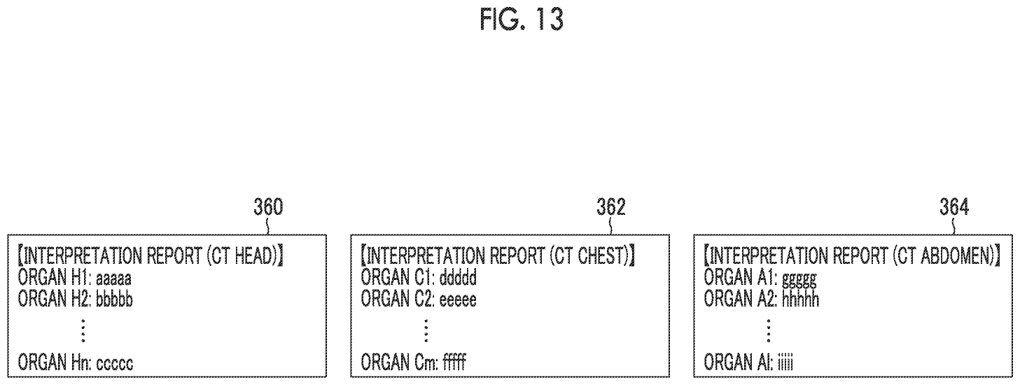

FIG. 13 is a schematic diagram showing a first example of a creation status of an interpretation report. FIG. 13 shows an example of an interpretation report of an “interpretation report created”, which corresponds to the first status. Regarding a medical image at an examination date and time for which the latest interpretation report should be created, in a case where an analysis result is obtained that a sentence related to an examination apparatus used in an examination, a sentence related to all defined parts, and a sentence related to all organs for each part are present in the interpretation report, the creation status of the interpretation report is determined to be an “interpretation report created”. The “interpretation report created” means that there are sentences for all organs for all parts of the interpretation report.

On the other hand, a case where the “interpretation report created” may include a case where there is a likelihood of changing the sentence and adding a sentence. The “interpretation report created” is distinguished from the completed interpretation report in which changes of and additions to the sentence do not occur. The completed interpretation report will be described later. An anatomical structure such as a blood vessel, a nerve, a bone, or a tendon may be applied to the organ referred to here.

In a case where the creation status of the latest interpretation report is determined to be an “interpretation report created”, all the medical images of the examination image group including the latest medical image to be interpreted correspond to the first status indicating an “interpretation report created”.

The medical image at the examination date and time for which the latest interpretation report should be created is a medical image generated in the latest examination, and may include a medical images to be interpreted and a past medical image to be compared with the medical image generated in the latest examination.

A head CT interpretation report 360 shown in FIG. 13 includes sentences for all organs included in the head regarding a medical image generated by imaging the head using a CT apparatus. Similarly, a chest CT interpretation report 362 includes sentences for all organs included in the chest regarding a medical image generated by imaging the chest using a CT apparatus. An abdominal CT interpretation report 364 includes sentences for all organs included in the abdomen regarding a medical image generated by imaging the abdomen using a CT apparatus.

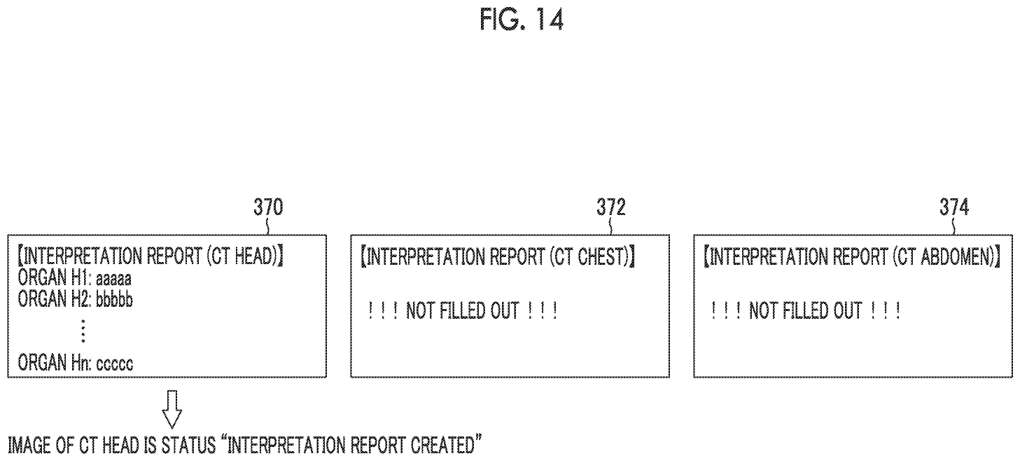

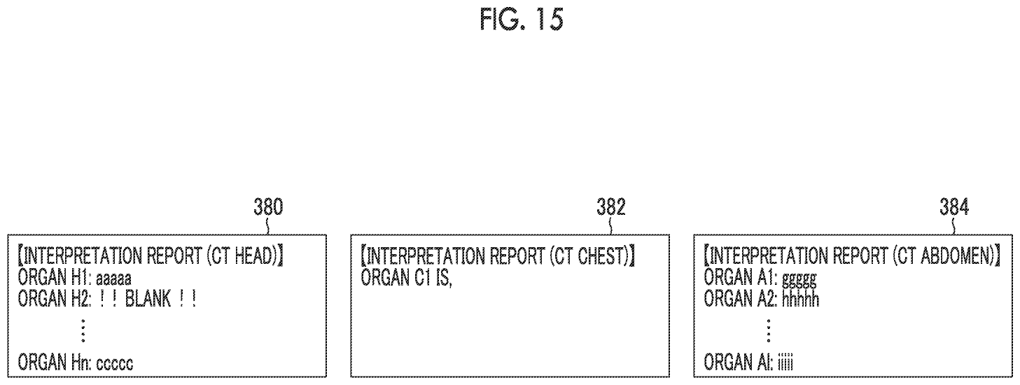

Note that the character n in the head CT interpretation report 360, the character m in the chest CT interpretation report 362, and the character l in the abdominal CT interpretation report 364 are any integers of 1 or more applied to dates. The character n, the character m, and the character l may have the same value or may have different values. The same applies to the character n shown in FIG. 14, and the character n and the character l shown in FIG. 15.

Further, aaaaa and the like in the head CT interpretation report 360, the chest CT interpretation report 362, the abdominal CT interpretation report 364, and the like are schematic representations of character strings representing sentences for an organ H1, and the like. The same applies to aaaaa and the like shown in FIGS. 14 and 15.

FIG. 14 is a schematic diagram showing a second example of a creation status of an interpretation report. FIG. 14 shows a modification example of the interpretation report in a case where it is determined to be an “interpretation report created”. A head CT interpretation report 370 shown in FIG. 14 includes descriptions for all organs. On the other hand, a chest CT interpretation report 372 and an abdominal CT interpretation report 374 have not been filled out.

In such a case, the chest CT interpretation report 372 and the abdominal CT interpretation report 374 are incomplete, and thus the interpretation report is incomplete as a whole, but the first status indicating the “interpretation report created” may be acquired for all medical images of the head CT. For example, the interpretation report shown in FIG. 14 may be created in a case where only the head is examined using a CT apparatus in the latest examination.

FIG. 15 is a schematic diagram showing a third example of a creation status of an interpretation report. FIG. 15 shows an example of an interpretation report corresponding to an incomplete interpretation report. Regarding the medical image at the examination date and time for which the latest interpretation report should be created, in a case where there is an unfilled item in at least one of the examination apparatus, the part, or the organ, the creation status of the interpretation report is determined to be an “interpretation report incomplete”.

A head CT interpretation report 380 shown in FIG. 15 includes a blank for the item related to an organ H2. Further, in a chest CT interpretation report 382, the sentence related to an organ C1 is not completed. On the other hand, an abdominal CT interpretation report 384 includes sentences for all the organs. In such a case, the interpretation report is incomplete as a whole, and the second status indicating that the interpretation report is incomplete is acquired for all medical images included in the examination.

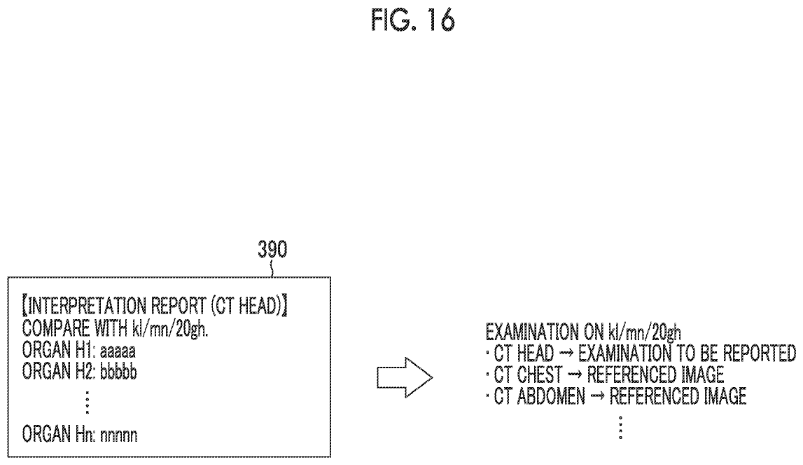

FIG. 16 is a schematic diagram showing a fourth example of a creation status of an interpretation report. FIG. 16 shows an example of an interpretation report corresponding to neither the completed interpretation report nor the incomplete interpretation report.

Among the past images, a medical image determined to be normal, a medical image that is not a target of comparative interpretation, and a medical image of a part different from the part that should be described in the latest interpretation report correspond to neither the “interpretation report created” nor the “interpretation report incomplete”.

The medical image determined to be normal may be mainly determined by a doctor or by CAD. In addition, CAD is an abbreviation for Computer-Aided Diagnosis. Examples of the medical image that is not a target of comparative interpretation include a medical image generated in an examination at a date and time different from the examination date and time of the medical image that is a target of comparative interpretation. Examples of the medical image of a part different from the part that should be described in the interpretation report include chest CT or abdominal CT medical images generated in the past examination in a case where the medical image generated in the latest examination targets only a head CT.

The latest head CT interpretation report 390 shown in FIG. 16 describes comparison with a head CT image generated on kl/mn/20gh, which is a past examination date. Then, the head CT image generated in the examination on kl/mn/20gh corresponds to a medical image that should be recorded in the interpretation report.

On the other hand, the chest CT image and the abdominal CT image generated in the past examination on kl/mn/20gh are medical images which are referred to, and correspond to neither the completed interpretation report nor the incomplete interpretation report. Note that g, h, k, l, m, and n shown in FIG. 16 are any integers of 1 or more applied to dates. g and the like may have the same value or may have different values.

### Procedure of Status Information Collection Method

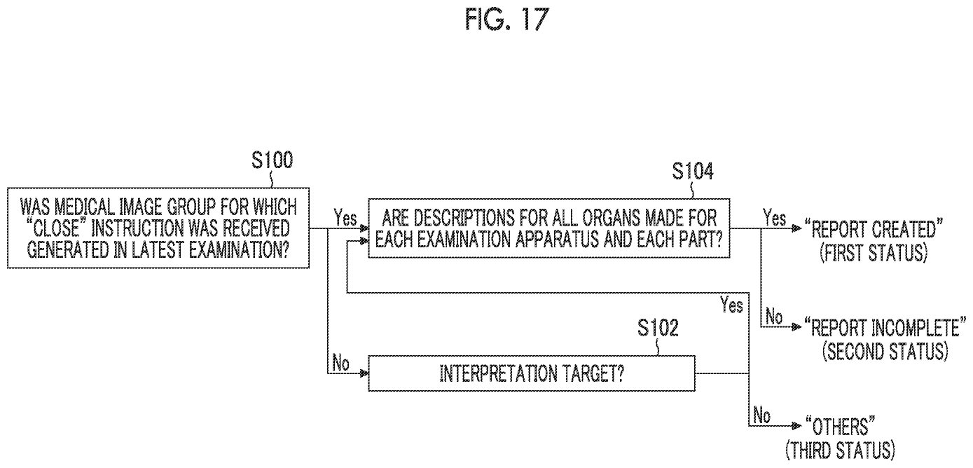

FIG. 17 is a flowchart showing a procedure of a status information collection method. Status information of the medical image displayed on the display device 26 is collected according to the following procedure. In a latest examination determination step S100, the status information collection unit 44 shown in FIG. 2 determines whether or not each of the one or more medical images acquired using the image acquisition unit 30 is included in the examination image group of the latest examination. That is, in the latest examination determination step S100, it is determined whether or not the imaging date and time of each acquired medical image matches the date and time of the latest examination.

A No determination is made for a medical image at an imaging date and time that does not match the date and time of the latest examination, and the process proceeds to an interpretation target determination step S102. On the other hand, in the latest examination determination step S100, a Yes determination is made for a medical image that matches the date and time of the latest examination, and the process proceeds to an interpretation report comparison determination step S104.

In the interpretation target determination step S102, the status information collection unit 44 refers to the analysis result of the interpretation report output from the interpretation report analysis unit 42 and determines whether or not the medical image for which status information is to be collected is a medical image to be interpreted. That is, in the interpretation target determination step S102, it is determined whether or not there is a description regarding a comparative image, such as the date and time, in the interpretation report to be referred to.

In the interpretation target determination step S102, in a case where it is determined that the medical image is not a medical image to be interpreted, a No determination is made, and the medical image corresponding to the No determination corresponds to the third status of “others”, which corresponds to neither the “interpretation report created” nor the “interpretation report incomplete”. On the other hand, in the interpretation target determination step S102, in a case where the medical image is a medical image to be interpreted, a Yes determination is made, and the process proceeds to the interpretation report comparison determination step S104. The interpretation target referred to here includes the past image to be compared with the current image.

In the interpretation report comparison determination step S104, the status information collection unit 44 refers to the analysis result of the interpretation report output from the interpretation report analysis unit 42, and determines whether or not there is a description for the examination apparatus, whether or not there are descriptions for all the parts for which a report is to be created, and whether or not there are descriptions for all the organs for each part.

In the interpretation report comparison determination step S104, in a case where there is no description for the examination apparatus, in a case where there is no description for some parts for which a report is to be created, or in a case where there is no description for some organs for each part, a No determination is made. In a case where the No determination is made, this corresponds to the second status indicating the “interpretation report incomplete”.

On the other hand, in a case where there is a description for the examination apparatus, in a case where there are descriptions for all the parts for which a report is to be created, and in a case where there are descriptions for all the organs for each part, a Yes determination is made. In a case where the Yes determination is made, this corresponds to the first status indicating the completed interpretation report.

Although FIG. 17 shows, as an example, an aspect in which status information is collected for all the acquired medical images, status information may be collected for some of the acquired medical images. For example, status information may be collected for a medical image for which a close instruction has been received, and status information may not be collected for a medical image for which a close instruction has not been received. The medical image for which a close instruction has been received referred to here may be a medical image included in the examination image group for which a close instruction has been received.

### Modification Example of Status Information Collection

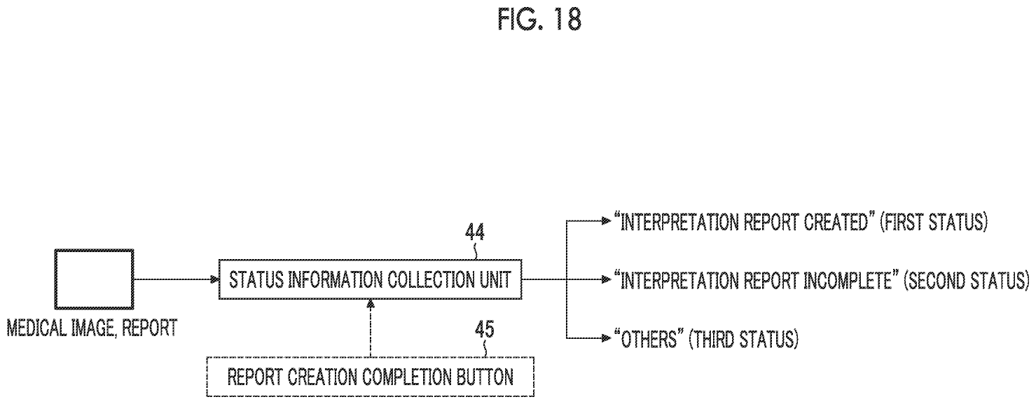

FIG. 18 is a schematic diagram showing a modification example of status information collection. The status information collection unit 44 shown in FIG. 18 may receive an input signal indicating completion of the interpretation report, and collect status information of the medical image based on the input signal indicating completion of the interpretation report. For example, in a case where a report creation completion button 45 is displayed on the display screen 100 shown in FIG. 5, and the report creation completion button 45 is operated, an input signal indicating completion of the creation of the interpretation report is transmitted to the status information collection unit 44. Completion of the creation of the interpretation report represents that the latest interpretation report includes sentences for all organs in all parts, and is stored as a completed interpretation report. In a case where the creation of the interpretation report is completed, processing such as correction and addition to the latest interpretation report may be prohibited.

In a case where an input signal indicating completion of the creation of the interpretation report is received, the status information collection unit 44 collects status information indicating the third status in the status information collection step S14 shown in FIG. 4, and in the processing determination step S18, processing of closing a target image without executing processing of confirming whether to close the target image is determined.

On the other hand, in a case where the status information collection unit 44 has not received an input signal indicating completion of the report, status information for the medical image for which a close instruction has been received is collected according to the procedure shown in FIG. 17.

According to the modification example of status information collection, it is possible to reduce the number of steps in the collection of status information, and it is possible to shorten the processing time of collecting the status information and to reduce the processing load.

### Collection of Status Information Using Learning Model

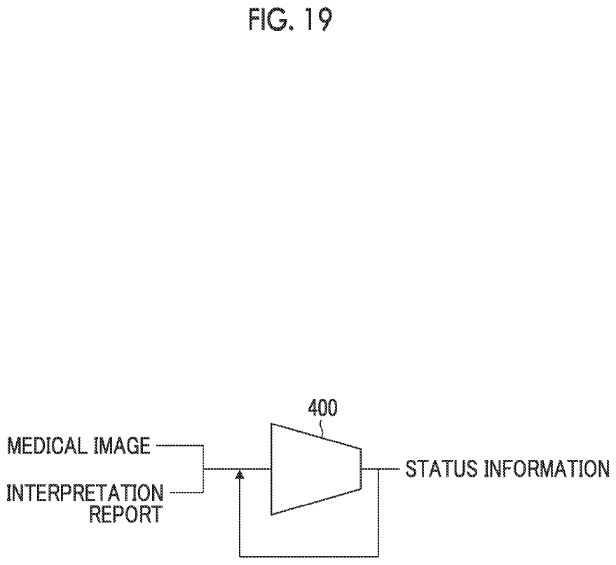

FIG. 19 is a schematic diagram of a learning model that performs collection of status information. A learning model 400 shown in FIG. 19 is a trained classification model that has been trained to collect status information according to the definition of the first status shown in FIGS. 13 and 14, the definition of the second status shown in FIG. 15, and the definition of the third status shown in FIG. 16. Deep learning or the like is applied to a learning method of the learning model 400.

That is, the learning model 400 is a trained learning model that has been trained to output the status information in a case where the medical image and the interpretation report are input, using learning data in which the medical image and the interpretation report are used as input data and the status information is used as correct answer data.

The learning model of the learning model 400 may be generated by performing learning using learning data in which the analysis result of the medical image and the analysis result of the latest interpretation report are used as input data and the status information is used as an output.

The trained learning model 400 shown in FIG. 19 may be applied to the status information collection unit 44 shown in FIG. 2. The status information collection unit 44 shown in FIG. 2 may perform relearning using the medical image and the interpretation report input to the status information collection unit 44 and the output status information as learning data.

### Specific Example of Processing Determined in Case Where Close Instruction is Received

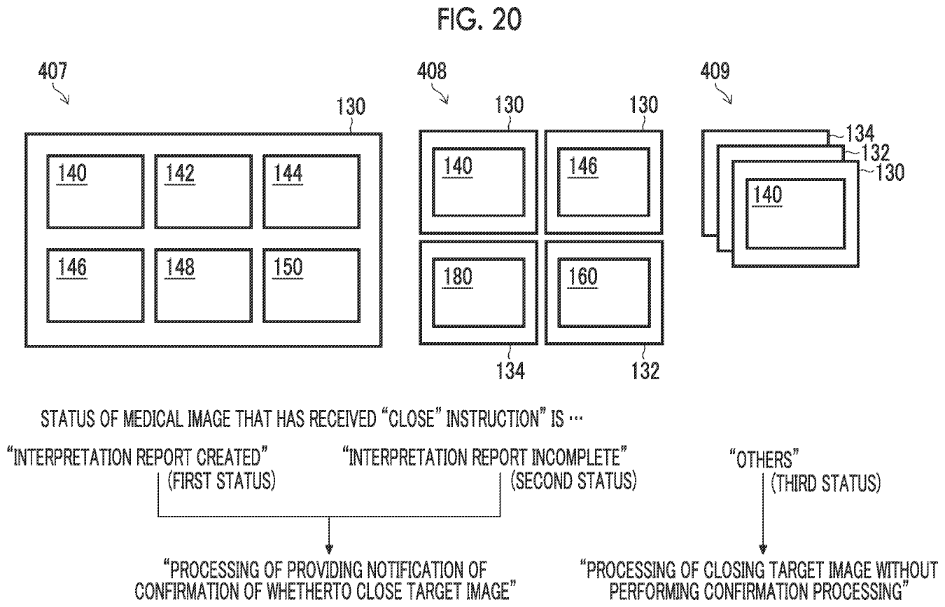

FIG. 20 is a schematic diagram showing a first example of determining processing for a close instruction. A first display example 407 shown in FIG. 20 is an aspect in which one examination image group 130 shown in FIG. 7 is displayed and a plurality of medical images 140 and the like included in the examination image group 130 are displayed.

A second display example 408 is an aspect in which a plurality of examination image groups 130 and the like shown in FIG. 8 are displayed and one or more medical images 140 and the like in each examination image group are displayed. A third display example 409 is an aspect in which one or more medical images 140 and the like included in one examination image group 130 shown in FIG. 9 are displayed and the other examination image group 134 and the like are stored without being displayed.

In a case where an instruction to close any one of the medical images is provided and the medical image for which a close instruction has been received corresponds to the first status or the second status, processing of providing notification of a confirmation of whether to close the medical image is determined, and the determined processing is executed.

On the other hand, in a case where the medical image for which a close instruction has been received corresponds to the third status, processing of closing the medical image without performing processing of providing notification of a confirmation of whether to close the medical image is determined, and the determined processing is performed.

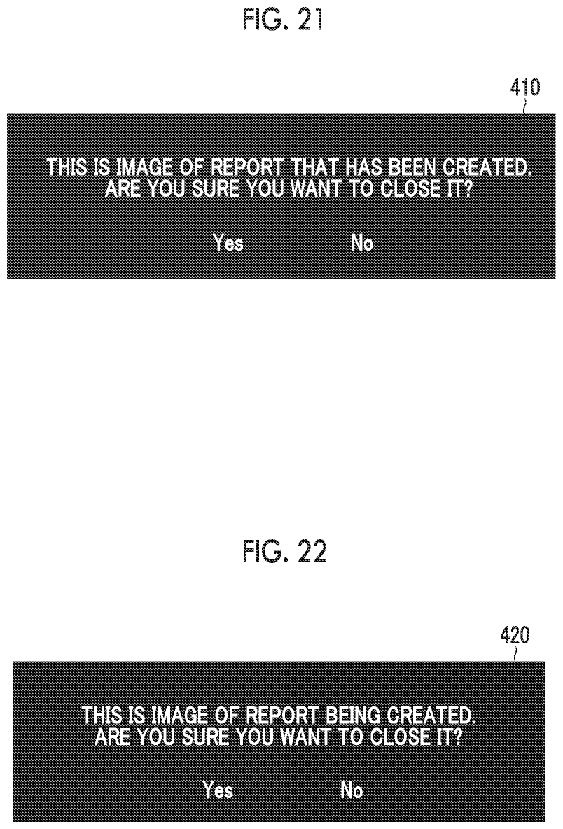

FIG. 21 is a schematic diagram showing an example of a pop-up. A pop-up 410 shown in FIG. 21 is displayed in a case where a close instruction for the medical image corresponding to the first status indicating a “report created” is received. A user may execute the close instruction by clicking Yes, or may stop the close instruction by clicking No.

FIG. 22 is a schematic diagram showing another example of a pop-up. A pop-up 420 shown in FIG. 22 is displayed in a case where a close instruction for the medical image corresponding to the second status is received. Similarly to the case where the pop-up 410 shown in FIG. 21 is displayed, the user can select to execute the closing processing or stop the closing processing. In a case where a close instruction for the medical image corresponding to the second status is received, the pop-up 320 shown in FIG. 12 may be displayed.

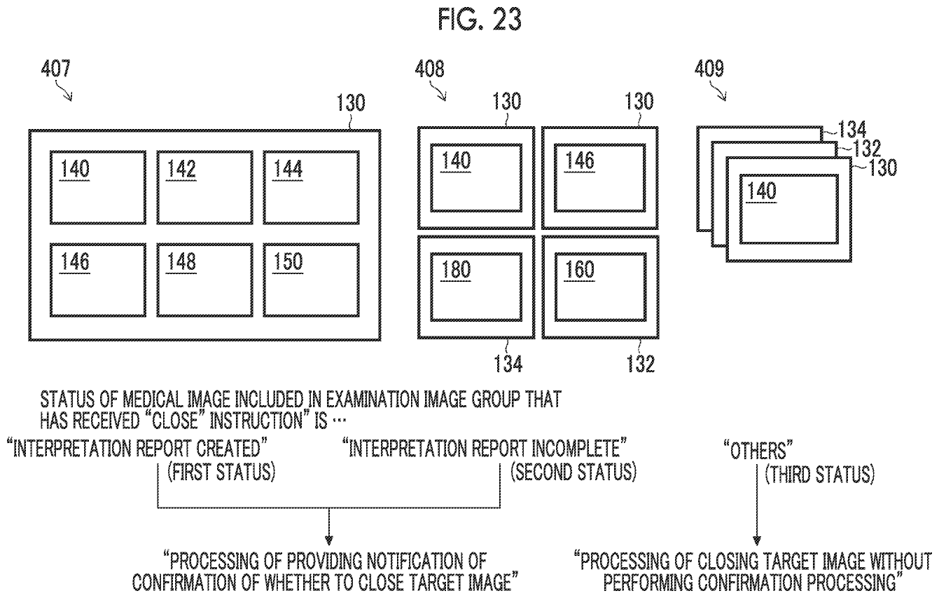

FIG. 23 is a schematic diagram showing a second example of determining processing for a close instruction. FIG. 23 shows an example of processing in a case where a close instruction for the examination image group is received. In a case where a close instruction for the examination image group is received, the same processing is determined and executed for all the medical images included in the examination image group.

In a case where any of the medical images included in the examination image group for which the closing processing has been received corresponds to the first status or the second status, processing of providing notification of a confirmation of whether to close the medical image is determined, and the determined processing is executed. On the other hand, in a case where any of the medical images included in the examination image group for which the closing processing has been received corresponds to the third status, processing of closing any of the medical images without performing processing of providing notification of a confirmation of whether to close any of the medical images is determined, and the determined processing is executed.

In a case where at least one of a medical image corresponding to the first status or a medical image corresponding to the second status and a medical image corresponding to the third status are present for a plurality of medical images included in the same examination image group, priority is given to the first status or the second status.

For example, in a case where a medical image corresponding to the first status and a medical image corresponding to the third status are present in the examination image group for which closing processing has been received, priority is given to the first status, and processing of providing notification of a confirmation of whether to close all medical images included in the examination image group is determined and executed.

In addition, in a case where a medical image corresponding to the second status and a medical image corresponding to the third status are present in the examination image group for which closing processing has been received, as in the case described above, processing of providing notification of a confirmation of whether to close all medical images included in the examination image group is determined and executed.

Further, in a case where a medical image corresponding to the first status, a medical image corresponding to the second status, and a medical image corresponding to the third status are present in the examination image group for which closing processing has been received, as in the case described above, processing of providing notification of a confirmation of whether to close all medical images included in the examination image group is determined and executed.

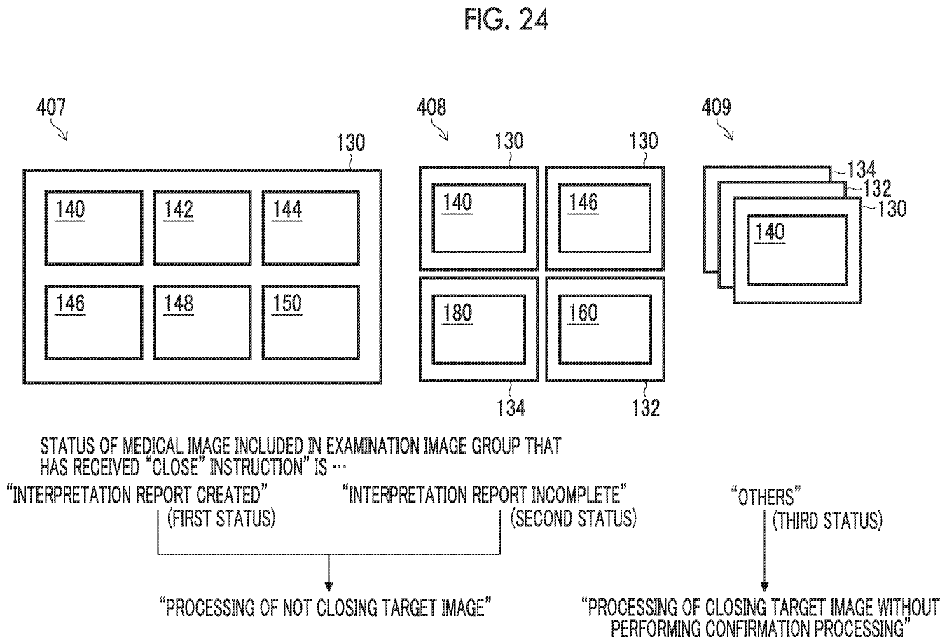

FIG. 24 is a schematic diagram showing a third example of determining processing for a close instruction. FIG. 24 shows another example of processing in a case where a close instruction for the examination image group is received. In the processing shown in FIG. 24, in a case where any status of the medical images included in the examination image group for which the closing processing has been received is the first status or the second status, processing of not closing the medical image is determined, and the determined processing is executed. In such a case, a pop-up indicating that processing of not closing the medical image has been determined may be displayed. Moreover, as in the pop-up 410 shown in FIG. 21, a button for selecting whether to execute the processing notified in response to the pop-up may be added.

On the other hand, in a case where any status of the medical images included in the examination image group for which the closing processing has been received is the third status, as in the case shown in FIG. 23 or the like, processing of closing the medical image without performing processing of providing notification of a confirmation of whether to close the medical image is determined, and the determined processing is executed. The processing of not closing the medical image described in the embodiment is an example of processing of continuing an open state of the medical image.

### Variation of Medical Images Displayed on Display Device

In the above-described embodiment, the two-dimensional slice image reconstructed from the three-dimensional raw data is exemplified as the medical image displayed on the display device 26, but a three-dimensional reconstructed image may be displayed.

### Effects of Embodiment

The information processing apparatus and the information processing method according to the embodiment can obtain the following effects.

[1]

Status information indicating the creation status of the interpretation report in the latest examination is collected for the medical image for which the close instruction has been received. The status information includes any of a first status indicating that the interpretation report has been created, a second status indicating that the interpretation report is incomplete, or a third status corresponding to neither the first status nor the second status. In a case where the medical image for which a close instruction has been received corresponds to the first status or the second status, processing of providing notification of a confirmation of whether to close the medical image is determined and is performed. Accordingly, execution of processing of erroneously closing a medical image for which an interpretation report in the latest examination is to be created is suppressed.

[2]

In a case where the medical image for which a close instruction has been received corresponds to the third status, an instruction to close the medical image without performing processing of providing notification of a confirmation of whether to close the medical image is provided. Accordingly, a medical image that is not important in creating the interpretation report in the latest examination is quickly closed.

[3]

In a case where processing of closing an examination image group including one or more medical images is received and any of the medical images included in the examination image group corresponds to the first status or the second status, processing of providing notification of a confirmation of whether to close the medical image is determined and is performed. Accordingly, execution of processing of erroneously closing a medical image for which an interpretation report in the latest examination is to be created is suppressed.

[4]

In a case where processing of closing an examination image group including one or more medical images is received and any of the medical images included in the examination image group corresponds to the first status or the second status, processing of not closing the medical image is determined and is executed. Accordingly, execution of processing of erroneously closing a medical image for which an interpretation report in the latest examination is to be created is suppressed.

[5]

In a case where any of the medical images included in the examination image group corresponds to the third status, an instruction to close any of the medical images without performing processing of providing notification of a confirmation of whether to close any of the medical images is determined and is performed. Accordingly, a medical image that is not important in creating the interpretation report in the latest examination is quickly closed.

[6]

The latest image, which is a medical image generated in the latest examination, corresponds to the first status or the second status. Accordingly, execution of processing that is erroneously closed on the latest image is suppressed.

[7]

Among past images that are medical images generated in past examinations, the past image to be compared with the latest image corresponds to the first status or the second status. Accordingly, execution of processing of erroneously closing a medical image to be compared with the latest image among past images is suppressed.

[8]

The interpretation report of the latest examination is analyzed, and the status of the medical image for which a close instruction is received is determined based on the examination date and time, part, and organ included in the interpretation report of the latest examination. Accordingly, status information reflecting the creation status of the interpretation report in the latest examination is collected.

[9]

In a case where an instruction indicating the completion of the interpretation report for the latest examination is received, the medical image for which the instruction indicating the completion of the interpretation report is received corresponds to the third status. Accordingly, it is possible to reduce the number of steps in the collection of status information, and it is possible to shorten the processing time of collecting the status information and to reduce the processing load.

[10]

The status information of the medical image for which the close instruction is received is collected using the trained learning model 400 that has been trained to collect the status information according to the definition of the first status, the definition of the second status, and the definition of the third status. Accordingly, high accuracy of the processing in collection of status information is achieved.

The technical scope of the invention is not limited to the scope described in the above embodiment. The configurations and the like in each embodiment can be appropriately combined between the respective embodiments without departing from the spirit of the present invention.

### EXPLANATION OF REFERENCES

10: hospital system
12: examination apparatus
12A: CT apparatus
12B: MRI apparatus
12C: PETCT apparatus
14: radiology information system
16: hospital information system
16A: automatic reception system
16B: electronic medical record system
16C: hospitalization/discharge management system
16D: medical accounting system
16E: pharmacy management system
16F: medical care reservation system
18: image storage communication system
20: report creation system
21: report creation terminal device
22: server device
23: database
24: display terminal
26: display device
27: input device
30: image acquisition unit
32: image processing unit
34: display signal output unit
40: input information reception unit
41: image analysis unit
42: interpretation report analysis unit
44: status information collection unit
45: report creation completion button
46: processing determination unit
48: processing execution unit
52: processor
54: computer-readable medium
56: communication interface
58: input/output interface
60: bus
62: memory
64: storage
70: image acquisition program
72: image processing program
74: display signal output program
80: input information reception program
82: interpretation report analysis program
83: image analysis program
84: status information collection program
86: processing determination program
88: processing execution program
100: display screen
102: title bar
104: information bar
106: toolbar
108: image display region
110: examination image group list
112: thumbnail image
120: medical image
122: medical image
124: medical image
126: medical image
130: examination image group
130A: folder
132: examination image group
134: examination image group
136: examination image group
140: medical image
142: medical image
144: medical image
146: medical image
148: medical image
150: medical image
200: medical image
202: medical image
204: medical image
206: medical image
208: medical image
210: medical image
212: medical image
214: medical image
216: medical image
300: interpretation report
320: pop-up
360: head CT interpretation report
362: chest CT interpretation report
364: abdominal CT interpretation report
370: head CT interpretation report
372: chest CT interpretation report
374: abdominal CT interpretation report
380: head CT interpretation report
382: chest CT interpretation report
384: abdominal CT interpretation report
390: head CT interpretation report
400: learning model
407: first display example
408: second display example
409: third display example
410: pop-up
420: pop-up
S10 to S24: each step of information processing method
S100 to S104: each step of status information collection method

What is claimed is:

1. An information processing apparatus comprising:
one or more processors; and
one or more memories in which instructions to be executed by the one or more processors are stored,
wherein the one or more processors are configured to:
receive an instruction to close one or more medical images displayed on a display device;
collect status information indicating any of a first status, a second status, or a third status, the first status being a status according to a creation status of a latest interpretation report for a first medical image that is a target of the instruction and indicating that the latest interpretation report has been created, the second status indicating that the latest interpretation report is incomplete, the third status corresponding to neither the first status nor the second status; and
determine processing according to the status information for the first medical image.

2. The information processing apparatus according to claim 1,
wherein the one or more processors are configured to, in a case where the status information of the first medical image indicates the first status or the second status, execute processing of providing notification of a confirmation of whether to close the first medical image, as the processing according to the status information.

3. The information processing apparatus according to claim 1,
wherein the one or more processors are configured to, in a case where the status information of the first medical image indicates the first status or the second status, execute processing of continuing an open state of the first medical image, as the processing according to the status information.

4. The information processing apparatus according to claim 1,
wherein the one or more processors are configured to, in a case where the status information of the first medical image indicates the third status, execute processing of closing the first medical image without performing processing of providing notification of a confirmation of whether to close the first medical image, as the processing according to the status information.

5. The information processing apparatus according to claim 1,
wherein the one or more processors are configured to receive an instruction to close an examination image group including the one or more medical images, as the instruction to close the one or more medical images displayed on the display device.

6. The information processing apparatus according to claim 5,
wherein the one or more processors are configured to:
in a case where the close instruction for the examination image group is received and the status information of the first medical image included in the examination image group indicates the first status or the second status, execute processing of providing notification of a confirmation of whether to close all the medical images included in the examination image group, as the processing according to the status information; and
in a case where the close instruction for the examination image group is received and the status information of the first medical image included in the examination image group indicates the third status, execute processing of closing all the medical images included in the examination image group without performing the processing of providing notification of the confirmation of whether to close all the medical images, as the processing according to the status information.

7. The information processing apparatus according to claim 5,
wherein the one or more processors are configured to:
in a case where the close instruction for the examination image group is received and the status information of the first medical image included in the examination image group indicates the first status or the second status, execute processing of continuing an open state of all the medical images included in the examination image group, as the processing according to the status information; and
in a case where the close instruction for the examination image group is received and the status information of the first medical image included in the examination image group indicates the third status, execute processing of closing all the medical images included in the examination image group without performing processing of providing notification of a confirmation of whether to close all the medical images, as the processing according to the status information.

8. The information processing apparatus according to claim 1,
wherein the one or more processors are configured to determine the status of the first medical image based on information represented by a character string input in a case of creating the latest interpretation report.

9. The information processing apparatus according to claim 7,
wherein the one or more processors are configured to determine the status of the first medical image by applying a trained learning model that has been trained to output the status information of the first medical image in response to inputs of the interpretation report for the first medical image and the first medical image using the latest interpretation report, the first medical image, and the status information of the first medical image as learning data.

10. The information processing apparatus according to claim 1,
wherein the one or more processors are configured to:
determine whether or not there is an interpretation report corresponding to the first medical image;
in a case where there is an interpretation report corresponding to the first medical image, determine the status information for the first medical image to be the first status; and
in a case where there is no interpretation report corresponding to the first medical image, determine the status information for the first medical image to be the second status.

11. The information processing apparatus according to claim 1,
wherein the one or more processors are configured to:
determine whether or not a character string is present in an interpretation report for an anatomical structure included in the first medical image;
in a case where the character string is present in the interpretation report for the anatomical structure included in the first medical image, determine the status information for the first medical image to be the first status; and
in a case where the character string is not present in the interpretation report for the anatomical structure included in the first medical image, determine the status information for the first medical image to be the second status.

12. The information processing apparatus according to claim 1,
wherein the one or more processors are configured to:
receive an input indicating completion of creation of an interpretation report; and
in a case where an input indicating completion of an interpretation report corresponding to the first medical image is received, determine the status information for the first medical image to be the third status.

13. The information processing apparatus according to claim 11,
wherein the one or more processors are configured to, in a case where it is determined that the status information for the first medical image indicates the third status, execute processing of closing the first medical image without performing processing of providing notification of a confirmation of whether to close the first medical image, as the processing according to the status information.

14. The information processing apparatus according to claim 1,
wherein the one or more processors are configured to determine the status of the first medical image based on an examination date and time at which the first medical image was generated.

15. An operation method of an information processing apparatus that performs information processing for a medical image to which a computer is applied, the operation method comprising:
by the information processing apparatus,
receiving a close instruction for one or more medical images displayed on a display device;
collecting status information indicating any of a first status, a second status, or a third status, the first status being a status according to a creation status of a latest interpretation report for a first medical image that is a target of the instruction and indicating that the interpretation report has been created, the second status indicating that the interpretation report is incomplete, the third status corresponding to neither the first status nor the second status; and
determining processing according to the status information for the first medical image.

16. A non-transitory, computer-readable tangible recording medium which records thereon a program causing, when read by a computer, the computer to implement:
a function of receiving a close instruction for one or more medical images displayed on a display device;
a function of collecting status information indicating any of a first status, a second status, or a third status, the first status being a status according to a creation status of an interpretation report for a first medical image that is a target of the instruction and indicating that the interpretation report has been created, the second status indicating that the interpretation report is incomplete, the third status corresponding to neither the first status nor the second status; and
a function of determining processing according to the status information for the first medical image.
